(19) United States
(12) Patent Application Publication
(10) Pub. No.: US 2022/0016344 A1
(43) Pub. Date: Jan. 20, 2022

(54) DOSAGE MANAGEMENT ASSISTANCE PROGRAM

(71) Applicant: Japan Science and Technology Agency, Kawaguchi-Shi (JP)

(72) Inventors: Toshiaki OHARA, Okayama-Shi (JP); Hiroshi SUITO, Sendai-Shi (JP); Yoshiki SUGITANI, Sakado-shi (JP)

(73) Assignee: Japan Science and Technology Agency, Kawaguchi-Shi (JP)

(21) Appl. No.: 17/382,853

(22) Filed: Jul. 22, 2021

Related U.S. Application Data
(63) Continuation of application No. PCT/JP2020/002305, filed on Jan. 23, 2020.

(30) Foreign Application Priority Data
Jan. 23, 2019 (JP) 2019-009333

(51) Int. Cl. A61M 5/172; G16H 20/17

(52) U.S. Cl. CPC A61M 5/1723; A61M 2005/14208; G16H 20/17

(57) ABSTRACT

The drug administration quantitative management assisting system includes an inputter and a calculator. The inputter receives, as input data, a time passed from previous drug administration to a patient and/or a value of biological materials in blood of the patient and/or a change of the value. The calculator calculates probabilities of drug administration to the patient as trinary determination of the dosage direction of STAY, UP or DOWN on the basis of a calculation model, and a first determination for determining the dosage direction, and a second determination for determining the dosage direction of UP or DOWN if the first determination is NON-STAY. The calculation model is prepared by machine learning using, as training data, the time passed from previous drug administration to a plurality of patients and/or the value of the biological material in blood of the plurality of patients and/or the changes of the value, and data indicating, as previous determination of the drug administration to the plurality of patients determined by doctors, any one of the dosage directions.

### TECHNICAL FIELD

The present invention relates to a drug administration quantitative management assisting system.

### BACKGROUND ART

A drug administration management assisting system has been disclosed, which includes an acquisition processor structured to acquire a treatment historical data relating to administration history of an anticancer drug, an extraction processor structured to extract, from the treatment historical data, specific historical data that is identical in terms of a patient and treatment with target prescription data being a target of processes and corresponding to prescription data of a session just prior to a session of the specific historical data being identical with the specific historical data in terms of administration time in the session, and an output processor structured to output the target prescription data and the specific historical data (for example, see Patent Literature 1).

### PATENT LITERATURE

[Patent Literature 1] JP2018-165867

### SUMMARY OF INVENTION

### Technical Problem

Chronic renal failure patients requiring dialysis are such that a level of erythropoietin (hereinafter, referred to as “EPO”), which is a hematogenous hormone secreted from kidney, is reduced as kidney function is deteriorated. In order to supplement this reduction, chronic renal failure patients are treated with administration of erythropoietic stimulating agent formulation (hereinafter, referred to as an “ESA formulation”). In this case, the patients would fall into anemia if an amount of the ESA formulation thus administered is not enough. Because anemia would result in immune deficiency and thus increase risks of acquiring cold and other diseases, anemia is an unfavorable prognostic factor. On the other hand, the ESA formulation is expensive and cost of medical care would be high if the ESA formulation is administered in an unnecessarily excessive amount. Furthermore, excessive administration of the ESA formulation would be a cause of headache, high blood pressure, blood vessel blockage of patients. Therefore, it is necessary to accurately control the amount of the ESA formulation administration.

Moreover, chronic renal failure patients are administered with an iron-containing formulation containing iron content as a material for red blood cells by injection or oral administration. Insufficient amount of iron-containing formulation would be a cause of anemia, while the iron-containing formulation administered in an excessive amount would be cytotoxic. Therefore, it is also necessary to accurately control the amount of the iron-containing formulation administered.

Appropriate management of a blood sugar level for post-cardiovascular surgery patients is very important for preventing complication and improving prognosis. In general, surgical invasion and cardiotonic drugs administered after surgeries, and the like are factors for increasing the blood sugar level. This would be a cause for increasing post-surgery mortality rates. On the other hand, low blood sugar levels lower than 70 mg/dL would be a cause of poor prognosis of patients. Therefore, it is considered as ideal that the blood sugar levels of patients are managed to be within a range of 120 to 180 mg/dL by continuous medication of insulin to the patients after surgeries.

Conventionally, the amounts of the ESA formulation and the iron-containing formulations to be administered have been determined by professional judgement of the medical specialists. That is, the medical specialists determine a dosage direction of “STAY”, “UP” or “DOWN” on the basis of their experiences, referring to various contents of patient's bloods and administration history so far. However, the number of such specialists is not so enough.

The same is also true for blood sugar level management of the post-cardiovascular surgery patients. That is, the amounts of insulin to be administered to post-cardiovascular surgery patients have been determined as appropriate by doctors and nurses on the basis of their experiences or tacit knowledges. The doctors and nurses determine a dosage direction of STAY, UP or DOWN on the basis of their experiences, referring to blood sugar level of patient's bloods and administration history so far. However, the number of such doctors and nurses is not so enough.

As a means for supporting such administration quantitative management under deficiency of the medical specialists, use of a calculation model prepared by machine learning can be considered to automatically determine the administration amount. However, machine learning in medical fields have the following problems, unlike general machine learning based on big data.

One of the problems is that training data available for the machine learning is not so much. Blood data and administration data of patients are personal data, and therefore, consents from the patients for the use of such data for machine learning are necessary. However, it is difficult to obtain consents from a large number of patients in reality. Moreover, there is such a technical problem that patient data once stored in electronic health records is difficult to extract and use as data for machine learning. Thus, the machine learning in medical fields should inevitably rely on a limited number of small data.

Another one of the problems is that a degree of correctness of the training data is not stable. In case where the administration amount is calculated out by machine learning, results of determinations made by doctors previously are necessary as training data. However, such results of determinations made by doctors may or may not be correct always, depending on a degree of individual doctor's skill and clinical cases of the patients. Thus, the machine learning in medical fields should inevitably rely on training data whose degree of correctness is not stable.

Thus, it is requested to realize a system capable of calculating out the administration amount by machine learning on the basis of training data that is small data with an unstable degree of correctness.

The administration management system described in Patent literature 1 is not one that contributes to solution of these problems.

The present invention was made in view of these problems, and an object of the present invention is to provide a system for assisting administration amount management for patients who are treated with drug administration and require appropriate quantity management of the drug administration.

### Solution to Problem

In order to solve the above problem, a drug administration quantitative management assisting system according to an embodiment of the present invention includes an inputter and a calculator. The inputter receives, as input data, a time passed from previous drug administration to a patient and/or a value of biological materials in blood of the patient and/or a change of the value. The calculator calculates out, from the input data, probabilities of drug administration to the patient as three dosage directions of STAY, UP and DOWN on the basis of the calculation model, and calculates out a first determination for determining the dosage direction of STAY or NON-STAY on the basis of the calculated probabilities of drug administration, and a second determination for determining the dosage direction of UP or DOWN if the first determination is NON-STAY. The calculation model is prepared by machine learning by using, as training data, the time passed from previous drug administration to a plurality of patients and/or the value of the biological material in blood of the plurality of patients and/or the change of the value, and data indicating, as previous determination of the drug administration to the plurality of patients determined by doctors, any one of dosage directions of STAY, UP or DOWN.

The drug administration quantitative management assisting system may further include a calculation model updater structured to update the calculation model to a new calculation model.

The training data may include data indicating whether previous determination of the dosage direction made by a doctor for the patient was STAY, UP or DOWN.

The training data may further include data of amounts of previous drug administration.

The training data does not need to include data of patients who had been infected with an infectious disease or had a surgery.

The patient may be a chronic renal failure patient, the value of the biological material in the blood may include a Hb level, a Ferritin level, and a TSAT level, and the change of the value may be a change of the Hb level, and a drug administered by the drug administration may be at least one of an ESA formulation or an iron-containing agent.

The value of the biological material in the blood may further include an MCV level and the change of the value further includes a change of the MCV level.

The calculation model may output an indication that a supply amount of EPO to the patient from outside of patient's body and a necessary amount are balanced, an indication that an EPO amount in the body is not sufficient, or an indication that the EPO amount in the body is excess, on the basis of the determination of the dosage direction of STAY, UP or DOWN.

The calculator may calculate out a third determination for determining the dosage direction of largely UP or slightly UP if the second determination is UP, and a fourth determination for determining the dosage direction of largely DOWN or slightly DOWN if the second determination is DOWN.

The patient may be a post-cardiovascular surgery patient, the value of the biological material in the blood may be a blood sugar level, and the drug administered by the drug administration may be insulin.

The calculation model may be structured to output an indication whether the insulin administration to the patient is enough, not enough, or excess, from the determination of the dosage direction of STAY, UP and DOWN.

Note that the present invention may also be effectively embodied as arbitrary combination of these constituent elements, a method, a device, a program, transitory or non-transitory recording medium storing the program therein, and an embodiment embodied by reciprocal replacements between systems or the like.

### Advantageous Effects of Invention

According to the present invention, it is possible to realize a system for calculating out an administration amount for patients who are treated with drug administration and require appropriate quantity management of the drug administration, the system determining the dosage direction of STAY, UP or DOWN.

### BRIEF DESCRIPTION OF DRAWINGS

FIG. 1 is a functional block diagram illustrating a drug administration quantitative management assisting system according to a first embodiment.

FIG. 2 is a schematic diagram illustrating a calculation model stored in a calculation model storage of the drug administration quantitative management assisting system of FIG. 1.

FIG. 3 is a flow diagram illustrating an operation of a calculator of a drug administration quantitative management assisting system according to a sixth embodiment.

FIG. 4 is a functional block diagram illustrating a drug administration quantitative management assisting system according to an eighth embodiment.

FIG. 5 is a graph illustrating consistency and inconsistency between results of determinations made by a drug administration quantitative management assisting system according to a seventh embodiment and results of determinations made by the medical specialists.

FIG. 6 is a graph illustrating consistency and inconsistency between results of determinations made by a drug administration quantitative management assisting system according to the seventh embodiment and results of determinations made by the medical specialists.

FIG. 7 is a flow diagram illustrating an operation of a calculator of a drug administration quantitative management assisting system according to a thirteenth embodiment.

### DESCRIPTION OF EMBODIMENTS

Hereinafter, the present invention will be described on the basis of preferable embodiments, referring to the drawings. In embodiments and modifications, like or equivalent constituent components and members are labelled in the same manners and repeating explanations thereof will be omitted where appropriate. Moreover, sizes of the members illustrated in the drawings may be magnified or demagnified as appropriate for the sake of easy understanding. Furthermore, the drawing may illustrate, omitting some of members not important in explaining embodiments. Moreover, terms including ordinal numbers such as first and second are used to explain various constituent components but, the terms are used only for distinguishing one constituent component from the other constituent component, but not to limit the constituent component by the terms.

Before explaining embodiments of the present invention concretely, finding on which the present invention was established will be described herein. As described above, in general the machine learning in the medical fields should be inevitably based on such training data that is small data with an unstable degree of correctness. As a result of studies, the present inventors found that, in case of learning an administration amount of an ESA formulation or an iron-containing formulation to chronic renal failure patients, accuracy of the learning can be improved by appropriately setting the training data to be inputted.

More specifically, the training data used for learning the administration amount of an ESA formulation or an iron-containing formulation is a hemoglobin level (hereinafter, referred to as “Hb”), a stored iron level (hereinafter, referred to as “Ferritin”), a functional iron level (referred to as “TSAT”) and a change of the Hb level in bloods of a plurality of chronic renal failure patients, and data indicating, as previous determination on drug administration to the plurality of patients by doctors, any one of dosage directions of STAY, UP or DOWN. With this configuration, it becomes possible to calculate out determination highly accurately on new drug administration as to the dosage direction of STAY, UP or DOWN with respect to the previous drug administration, by inputting the Hb level, the Ferritin level, TSAT level, and the change of the Hb level in bloods of patients.

The input data may further include a mean corpuscular volume (hereinafter, referred to as “MCV) level, and a change of the MCV level. A calculation model may be prepared by machine learning using, as training data, the Hb level, the MCV level, the TSAT level, the Ferritin level, the change of the Hb level, and the change of the MCV level in bloods of the plurality of chronic renal failure patients, and the data indicating, as the determination on previous drug administration to the plurality of patients by doctors, any one of dosage directions of STAY, UP or DOWN.

### First Embodiment

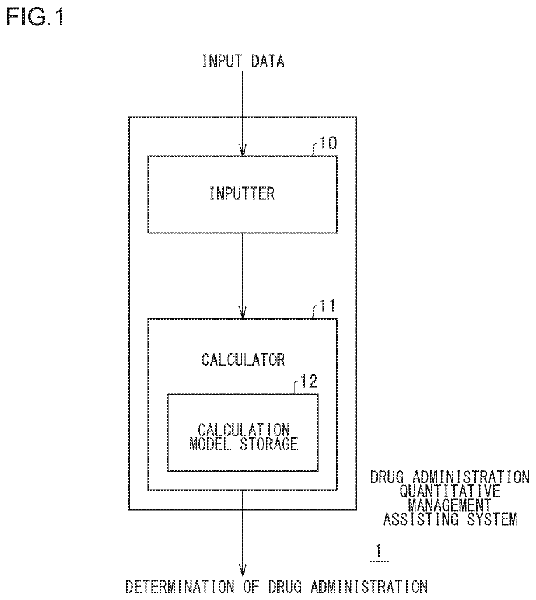

FIG. 1 is a functional block diagram illustrating a drug administration quantitative management assisting system 1 according to a first embodiment of the present invention. The drug administration quantitative management assisting system 1 includes an inputter 10 and a calculator 11. The calculator 11 includes a calculation model storage 12.

The inputter 10 is structured to receive input of an Hb level, a Ferritin level, and a TSAT level, and a change of the Hb level in blood of a chronic renal failure patient as input data. The input data is transmitted to the calculator 11.

The calculator 11 is structured to calculate out, from the input data received from the inputter 10, trinary determination of drug administration to the chronic renal failure patient as to the dosage direction of STAY, UP or DOWN with respect to the previous drug administration, on the basis of a calculation model stored in the calculation model storage 12. More specifically, the calculator 11 inputs, into the calculation model, the input data received from the inputter 10, thereby obtaining probabilities of the dosage direction of STAY, UP or DOWN for being determined as the determination of the drug administration. The calculator 11 calculates out the dosage direction of STAY, UP or DOWN, on the basis of these probabilities.

The calculation model stored in the calculation model storage 12 is prepared by machine learning. Training data used for the machine learning includes the Hb level, the Ferritin level, the TSAT level, the change of the Hb level in bloods of a plurality of chronic renal failure patients, and data indicating, as previous determination on drug administration to the plurality of patients by doctors, any one of dosage directions of STAY, UP or DOWN.

The calculation model stored in the calculation model storage 12 may be structured to receive the input of the Hb level, the Ferritin level, the TSAT level, and the change of the Hb level, and output the probabilities of the dosage direction of STAY, UP or DOWN for being determined as the determination of the drug administration.

Instead of the probabilities of the dosage direction of STAY, UP or DOWN for being determined as the determination of the drug administration, the calculation model may be structured to output an indication that a supply amount of EPO to the patient from outside of patient's body and a necessary amount are balanced, an indication that an EPO amount in the body is not sufficient, or an indication that the EPO amount in the body is excess, on the basis of the determination. As described above, the ESA formulation is administered if the EPO is not sufficient. Thus, the determinations of the dosage direction of STAY, UP or DOWN by the calculation model correspond to the state that the supply amount of EPO to the patient from outside of patient's body and the necessary amount are balanced, the state that the EPO amount in the body is not sufficient, and the state that the EPO amount in the body is excess, respectively. That is, the calculation model may be structured to output the indication that the supply amount of EPO to the patient from outside of patient's body and the necessary amount are balanced, the indication that the EPO amount in the body is not sufficient, or the indication that the EPO amount in the body is excess. For example, a doctor can find out a clinical state of the patient by looking at results of the output.

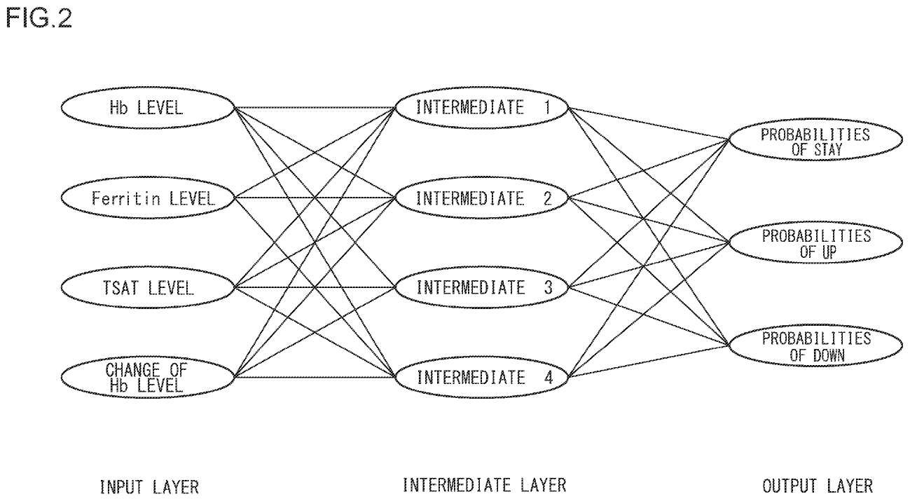

FIG. 2 is a schematic diagram illustrating an example of a calculation model stored in a calculation model storage 12. An input layer receives input of an Hb level, a Ferritin level, and a TSAT level, and a change of the Hb level in blood of a chronic renal failure patient for whom determination of the drug administration is necessary. A network including an intermediate layer stores therein the calculation model prepared by the machine learning. The calculation is performed by using the calculation model, thereby to output to an output layer the probabilities of the dosage direction of STAY, UP or DOWN.

According to the present invention, it is possible to realize a drug administration quantitative management assisting system for calculating out the dosage direction of STAY, UP or DOWN to a chronic renal failure patient.

### Second Embodiment

A drug administration quantitative management assisting system 1 according to a second embodiment of the present invention will be described with reference to FIG. 1. The drug administration quantitative management assisting system 1 includes an inputter 10 and a calculator 11. The calculator 11 includes a calculation model storage 12.

The inputter 10 is structured to receive input of an Hb level, an MCV level, a Ferritin level, a TSAT level, a change of the Hb level, and a change of the MCV level in blood of a chronic renal failure patient as input data. The input data is transmitted to the calculator 11.

The calculator 11 is structured to calculate out, from the input data received from the inputter 10, trinary determination of drug administration to the chronic renal failure patient as to the dosage direction of STAY, UP or DOWN with respect to the previous drug administration, on the basis of a calculation model stored in the calculation model storage 12. More specifically, the calculator 11 inputs, into the calculation model, the input data received from the inputter 10, thereby obtaining probabilities of the dosage direction of STAY, UP or DOWN for being determined as the determination of the drug administration. The calculator 11 calculates out the trinary determination of the drug administration as to the dosage direction of STAY, UP or DOWN, on the basis of these probabilities.

The calculation model stored in the calculation model storage 12 is prepared by machine learning. Training data used for the machine learning includes the Hb level, the MCV level, the Ferritin level, the TSAT level, the change of the Hb level, and the change of the MCV level in bloods of a plurality of chronic renal failure patients, and data indicating, as previous determination on drug administration to the plurality of patients by doctors, any one of dosage directions of STAY, UP or DOWN.

The calculation model stored in the calculation model storage 12 receives the input of the Hb level, the MCV level, the Ferritin level, the TSAT level, the change of the Hb level, and the change of the MCV level, and outputs the probabilities of the dosage direction of STAY, UP or DOWN for being determined as the determination of the drug administration.

According to the present invention, it is possible to realize a drug administration quantitative management assisting system for calculating out the dosage direction of STAY, UP or DOWN to a chronic renal failure patient by additionally including the MCV level and the change of the MCV level as additional training data to the training data of the first embodiment.

### Third Embodiment

The training data of a drug administration quantitative management assisting system 1 according to a third embodiment of the present invention includes data indicating, as previous determination of the drug administration made by a doctor for the patient, any one of dosage directions of STAY, UP or DOWN. The third embodiment is configured identically with the first and second embodiments except the above.

As a result of studies by the present inventors, it was found out that, immediately after the amount of the drug administration is changed, the next drug administration is often determined to be STAY so as to observe how the drug administration will go. Therefore, the training data for preparing the calculation model may additionally include the data indicating, as previous determination made by a doctor for a last drug administration to the patient, any one of dosage directions of STAY, UP or DOWN, in order to improve the accuracy of the calculation model.

In general, patients under dialysis are tested by drawing blood once in one or two weeks, and amounts of the drug administration are determined according to results of the test every time the test is done. Therefore, the aforementioned “determination made by a doctor for a last drug administration to the patient” can be considered as being substantially “determination made by a doctor for the drug administration to the patient one or two weeks before.”

According to the present embodiment, it is possible to realize a drug administration quantitative management assisting system capable of calculating out determination of drug administration with a high accuracy.

### Fourth Embodiment

Training data of a drug administration quantitative management assisting system 1 according to a fourth embodiment of the present invention further includes data of amounts of previous drug administration. The fourth embodiment is configured identically with the first and second embodiments except the above.

Note that the “data of the amount of the previous drug administration” may be considered as being substantially “data of the amount of the drug administration one or two weeks before herein”, like the third embodiment.

According to the present embodiment, it is possible to realize a drug administration quantitative management assisting system capable of calculating out correct determination of drug administration.

### Fifth Embodiment

Training data of a drug administration quantitative management assisting system 1 according to a fifth embodiment of the present invention further includes data indicating whether the amount of the previous drug administration was 0 or not. The fifth embodiment is configured identically with the first and second embodiments except the above.

As a result of the studies by the present inventors, it was found that, in case where the amount of the previous drug administration was 0, it is difficult to determine to be STAY. Therefore, the training data for preparing the calculation model may additionally include data indicating whether the amount of the previous drug administration was 0 or not, in order to improve the accuracy of the calculation model for determining how much the amount of the drug administration will be from the amount of the drug administration of 0.

Note that the “data indicating whether the amount of the previous drug administration was 0 or not” may be considered as being substantially “data of the amount of the drug administration one or two weeks before herein was 0 or not,” as in the third embodiment.

According to the present embodiment, it is possible to realize a drug administration quantitative management assisting system capable of calculating out determination of drug administration with a high accuracy.

### Sixth Embodiment

A calculator 11 of a drug administration quantitative management assisting system 1 according to a sixth embodiment of the present invention is structured to calculate out, on the basis of the probabilities of the dosage direction of STAY, UP or DOWN obtained by the calculation model stored in a calculation model storage 12, a first determination for determining the dosage direction of STAY or NON-STAY, and

a second determination for determining the dosage direction of UP or DOWN if the first determination is NON-STAY.

As described above, the calculation model storage 12 is structured to output the probabilities of the three dosage directions of the drug administration such as STAY, UP and DOWN.

In case where results obtained from a calculation model prepared from machine learning are binary (for example, in case where the drug administration is determined as to the dosage direction of UP or DOWN), the model can be evaluated in terms of operation by using a receiver operating characteristic curve (ROC curve) or the like. This can improve accuracy of final calculation results. However, in case where trinary results are obtainable from the calculation model, it is difficult to evaluate the model accurately. This is because, in case where the technique for the ROC curve is expanded for the trinary determination, it is necessary to set two thresholds, but it is difficult to find out an optimum solution with such two thresholds changed at the same time.

As a result of studies, the present inventors found that, in case where the drug administration is determined by three dosage direction of STAY, UP or DOWN, the determination of the dosage direction of STAY or NON-STAY (hereinafter, referred to as the “first determination”) and the determination of the dosage direction of UP or DOWN if the first determination is NON-STAY (hereinafter, referred to as the “second determination”) are different from each other in terms of characteristics of the determinations. That is, the first determination is relatively difficult but the second determination is relatively easy if the first determination has been already made. Based on this finding, the accuracy of the drug determination can be improved with this configuration that the trinary determination is carried out by two stages including making the first determination and making the second determination thereafter, as described above.

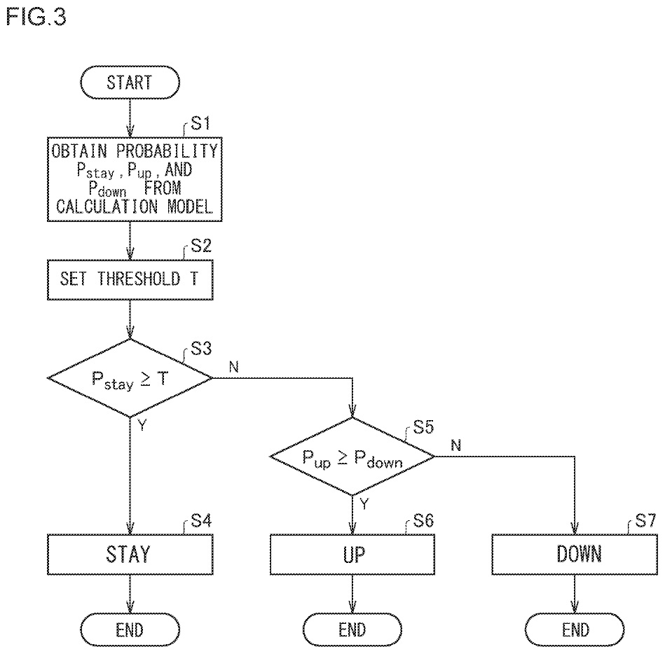

FIG. 3 is a flow diagram illustrating an operation of a calculator 11 of the drug administration quantitative management assisting system 1 according to the sixth embodiment.

At step S1, the calculator 11 obtains a probability P_{stay} of STAY, a probability Pᵤₚ of UP, and a probability P_{down} of DOWN in regard to the drug administration determination from the calculation model stored in the calculation model storage 12. Note that P_{stay}+Pᵤₚ+P_{down}=1.

At step S2, the calculator 11 sets a threshold T for the first determination. Note that 0<T<1. T=0 means that the determination is always STAY, while T=1 means that the determination is always to UP or DOWN.

At step S3, the calculator 11 makes the first determination, that is, determines STAY or NON-STAY. More specifically, the calculator 11 determines whether or not P_{stay}≥T.

If the first determination is positive at step S3, the process goes to step S4.

At step S4, the calculator 11 outputs a result of the determination that the dosage direction is STAY, and the process ends.

If the first determination is negative at step S3, the process goes to step S5.

At step S5, the calculator 11 makes the second determination, that is, determines UP or DOWN. More specifically, the calculator 11 determines whether or not Pᵤₚ≥P_{down}.

If the second determination is positive at step S5, the process goes to step S6.

At step S6, the calculator 11 outputs a result of the determination that the dosage direction is UP, and the process ends.

If the second determination is negative at step S5, the process goes to step S7.

At step S7, the calculator 11 outputs a result of the determination that the dosage direction is DOWN, and the process ends.

According to the present embodiment, it is possible to realize a drug administration quantitative management assisting system capable of calculating out determination of drug administration with a high accuracy.

### Seventh Embodiment

Training data of a drug administration quantitative management assisting system 1 according to a seventh embodiment of the present invention does not include data of patients who had been infected with an infectious disease or had a surgery. The seventh embodiment is configured identically with the first and second embodiments except the above.

As a result of the studies by the present inventors, it was found that the accuracy of the learning would be lowered if the training data included such data of the patients who had been infected with an infectious disease or had a surgery. Thus, by excluding from the training data the data of the patients who had been infected with an infectious disease or had a surgery, the accuracy of the calculation model can be improved.

According to the present embodiment, it is possible to realize a drug administration quantitative management assisting system capable of calculating out determination of drug administration with a high accuracy.

### Eighth Embodiment

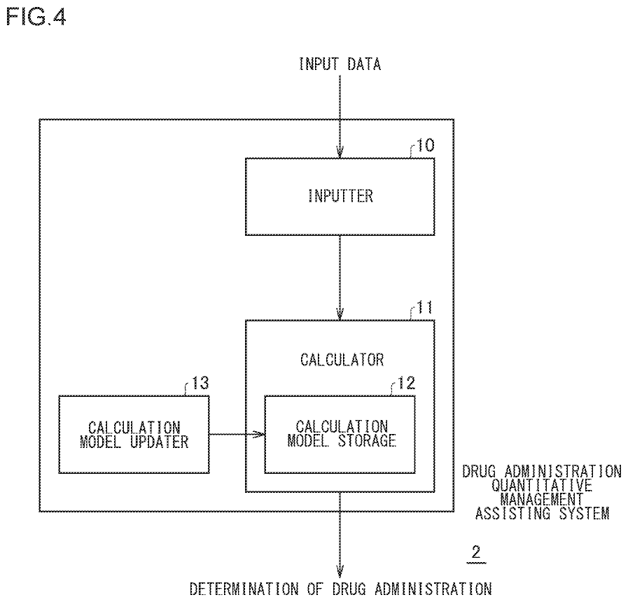

FIG. 4 is a functional block diagram illustrating a drug administration quantitative management assisting system 2 according to an eighth embodiment of the present invention. A drug administration quantitative management assisting system 2 includes a calculation model updater 13 structured to update the calculation model to a new calculation model. The other configurations of the drug administration quantitative management assisting system other than the above are identical with the configurations of the drug administration quantitative management assisting system 1 of FIG. 1 and the configuration of the second embodiment.

The calculation model prepared once can be updated to a calculation model by performing machine learning with additional training data, thereby being updated to a calculation model capable of outputting more correct results. By providing such updated new calculation model to the calculation model updater 13 periodically or as needed, the calculation model stored in the calculation model storage 12 is updated. With this configuration, the drug administration determination can be more correct.

According to the present embodiment, it is possible to upgrade the drug administration quantitative management assisting system to one capable of calculating out determination of drug administration with a higher accuracy.

### Verification 1

To verify applicability of the present invention to clinical uses, a determination of drug administration calculated by a drug administration quantitative management assisting system according to the present invention was compared with a determination made by a medical specialist. The verification was conducted by using a set of data for verification about the dosage direction of STAY, UP or DOWN of drug administration of an ESA formulation.

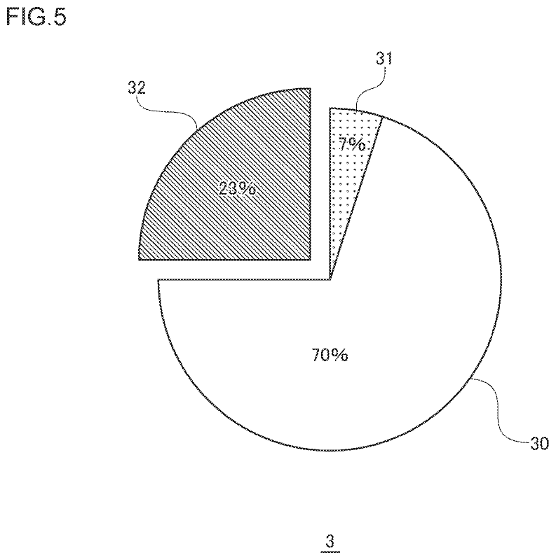

FIG. 5 is a graph 3 illustrating consistency and inconsistency between results of determinations in the following embodiment and results of determinations made by the medical specialists. That is, this embodiment is a drug administration quantitative management assisting system having the elements of the first embodiment: The input data includes an MCV level and a change of the MCV level; The training data includes data indicating whether previous determination of the drug administration made by a doctor for the patient was STAY, UP or DOWN, the data of the amount of the previous drug administration, and data indicating whether the amount of the previous drug administration was 0 or not. A calculator is structured to calculate out, on the basis of the probabilities of the dosage direction of STAY, UP or DOWN obtained from the calculation model, a first determination for determining the dosage direction of STAY or NON-STAY, and a second determination for determining the dosage direction of UP or DOWN if the first determination is NON-STAY. The training data does not include data of patients who had been infected with an infectious disease or had a surgery. Here, a region 30 is a ratio of cases where the determination of the drug administration quantitative management assisting system 1 matched with the determination made by the medical specialist, within the results of the dosage direction of STAY. A region 31 is a ratio of cases where the determination of the drug administration quantitative management assisting system 1 matched with the determination made by the medical specialist, within the results of the dosage direction of UP or DOWN. A region 32 is a ratio of cases where the determination of the drug administration quantitative management assisting system 1 did not match with the determination made by the medical specialist.

From FIG. 5, it can be understood that the determination of the drug administration quantitative management assisting system 1 matched with the determination made by the medical specialist by 77%, summing up the regions 30 and 31. Meanwhile, it can be understood that the determination of the drug administration quantitative management assisting system 1 did not match with the determination made by the medical specialist by 23%.

FIG. 5 illustrates simple consistency and inconsistency between results of determinations made by a drug administration quantitative management assisting system 1 and results of determinations made by the medical specialists. However, it should be noted that the unmatched cases include apparently unmatched cases in which the determinations are unmatched due to different timings of making the determinations between the drug administration quantitative management assisting system 1 and the medical specialist, more specifically, due to earlier determination days for the drug administration quantitative management assisting system 1 than the medical specialist.

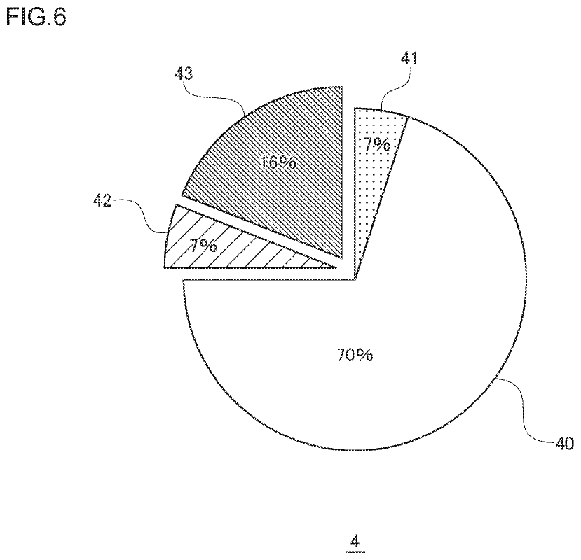

FIG. 6 is a graph 4 illustrating consistency and inconsistency between results of determinations made by a drug administration quantitative management assisting system according to the above-described embodiment and results of determinations made by the medical specialists. Note that FIG. 6 illustrates the regions of FIG. 5 in details. A region 40 is a ratio of cases where the determination of the drug administration quantitative management assisting system 1 dated identically with and matched with the determination made by the medical specialist, within the results of the dosage direction of STAY. A region 41 is a ratio of cases where the determination of the drug administration quantitative management assisting system 1 dated identically with and matched with the determination made by the medical specialist, within the results of the dosage direction of UP or DOWN. A region 42 is a ratio of cases where the determination of the drug administration quantitative management assisting system 1 dated earlier than and matched with the determination made by the medical specialist. A region 43 is a ratio of cases where the determination of the drug administration quantitative management assisting system 1 did not match with the determination made by the medical specialist, regardless of the dates of determination of the drug administration quantitative management assisting system 1 and the medical specialist.

From FIG. 6, it can be understood that the determination of the drug administration quantitative management assisting system 1 matched with the determination made by the medical specialist by 83%, summing up the regions 40, 41, and 42. Meanwhile, it can be understood that the determination of the drug administration quantitative management assisting system 1 did not match with the determination made by the medical specialist by 17%.

As a result of further detailed verification on each cases in the region 43 in FIG. 6, it was found that the region 43 includes some cases where the determination made by the drug administration quantitative management assisting system 1 was clearly wrong, as well as some cases where the determination made by the medical specialist was wrong but the determination made by the drug administration quantitative management assisting system 1 was correct, and some cases where it is not clear which one of the determinations was correct. A ratio of cases where the determination of the drug administration quantitative management assisting system 1 was clearly wrong was about 13% of the whole cases. That is, it was found that the drug administration quantitative management assisting system 1 could calculate out the drug administration amount with about 87% correctness with respect to the determinations made by the medical specialist.

From the results of the verification as above, the drug administration quantitative management assisting system 1 according to the present invention can be considered as being capable of determining the drug administration with accuracy sufficiently applicable to the clinical uses.

### Verification 2

In order to verify a difference in effect between the first embodiment (in which the training data and the input data do not include the MCV level and the change of the MCV level) and the second embodiment (in which the training data and the input data include the MCV level and the change of the MCV level), the cases of actual patients were verified. The learning data used herein was such that the number of patients was 131 and the number of targeted weeks was 6080. Moreover, evaluation data used herein was such that the number of patients was 87 and the number of targeted weeks was 1857. The result was as below.

(Cases 1) Cases where the determination of the drug administration quantitative management assisting system 1 dated identically with and matched with the determination made by the medical specialist. The cases were 80% in the first embodiment and 77% in the second embodiment.

(Cases 2) Cases 1 and cases where the determination of the drug administration quantitative management assisting system 1 dated earlier than and matched with the determination made by the medical specialist. The cases were 86% in the first embodiment and 84% in the second embodiment.

In either case, the first embodiment achieved slightly better results in terms of matching probabilities. This result has not been explained clearly yet, but for example, anemia has some factors not noticeable based on MCV, and this would have resulted in such a possibility that the result was better without MCV. Moreover, the present invention uses a probabilistic method, the degree of correction would vary among trials. The degree of correctness above is the best one among several trials. The difference appeared here depending on whether with or without the MCV is as much as a range of variations appearing time to time, and thus it can be said that there is not substantial difference between with or without the MCV. In either case, the first or second embodiment can be considered as being capable of performing drug administration determination sufficiently applicable to clinical uses.

### Verification 3

Both of the verifications 1 and 2 were such that the preparation of the learning data of the drug administration quantitative management assisting system and the determination made by the medical specialist were carried out in the same medical institution. In comparison with these, verification for verifying effectiveness of a case where the preparation of the learning data of the drug administration quantitative management assisting system and the determination made by the medical specialist were carried out in different medical institutions. Here, the second embodiment (in which the training data and the input data include the MCV level and the change of the MCV level) was used.

Hospital A: a hospital in which the preparation of the learning data of the drug administration quantitative management assisting system was carried out (the learning data was such that the number of patients was 131 and the number of targeted weeks was 6080. Evaluation data was such that the number of patients was 87 and the number of targeted weeks was 1857).

Hospital B: a hospital which did not participated in the preparation of the learning data of the drug administration quantitative management assisting system (evaluation data was such that the number of patients was 16 and the number of targeted weeks was 298. (note: the learning data used herein was the one prepared in the hospital A)

Here, the matching rates of the determinations of the medical specialist in the hospital A and the hospital B with the determination of the drug administration quantitative management assisting system were as below.

(Cases 3) Cases where the determination of the drug administration quantitative management assisting system 1 dated identically with and matched with the determination made by the medical specialist. Hospital A: 77%, Hospital B: 72%

(Cases 4) Cases 3 and cases where the determination of the drug administration quantitative management assisting system 1 dated earlier than and matched with the determination made by the medical specialist. Hospital A: 84%, Hospital B: 81%

In either case, the matching rate with the determination made by the medical specialist at the hospital A in which the learning data was prepared was higher, but the matching rate with the determination made by the medical specialist at the hospital B was sufficient. From these, it can be understood that the drug administration quantitative management assisting system can make determination of drug administration sufficiently generally similar to determination made by the medical specialists, regardless of whether the medical specialists have participated in the preparation of the learning data.

### Ninth Embodiment

A drug administration quantitative management assisting system 1 according to a ninth embodiment of the present invention will be described with reference to FIG. 1. The drug administration quantitative management assisting system 1 includes an inputter 10 and a calculator 11. The calculator 11 includes a calculation model storage 12.

The inputter 10 is structured to receive, as input data, blood sugar levels in blood, insulin administration amounts, and time periods passed from the end of the cardiovascular surgery to the insulin administration of a post-cardiovascular surgery patients. The input data is transmitted to the calculator 11.

The calculator 11 is structured to calculate out, from the input data received from the inputter 10, trinary determination of the insulin administration to the post-cardiovascular surgery patients as to the dosage direction of UP or DOWN with respect to the previous drug administration, on the basis of a calculation model stored in the calculation model storage 12. More specifically, the calculator 11 inputs, into the calculation model, the input data received from the inputter 10, thereby obtaining probabilities of the dosage direction of STAY, UP or DOWN for being determined as the determination of the insulin administration. The calculator 11 calculates out the trinary determination of the insulin administration as to the dosage direction of STAY, UP or DOWN, on the basis of these probabilities.

The calculation model stored in the calculation model storage 12 is prepared by machine learning. The training data used herein includes blood sugar levels in blood, insulin administration amounts, time periods passed from the end of the cardiovascular surgery to the insulin administration of a post-cardiovascular surgery patients, and data indicating, as determination of the previous insulin administration to the patients by doctors, any one of dosage directions of STAY, UP or DOWN.

The calculation model stored in the calculation model storage 12 receives the blood sugar levels in blood, the insulin administration amounts, and the time periods passed from the end of the cardiovascular surgery to the insulin administration of a post-cardiovascular surgery patients, and outputs probabilities of the dosage direction of STAY, UP or DOWN in regard to the determination of the insulin administration.

Instead of the probabilities of the dosage direction of STAY, UP or DOWN for being determined as the determination of the insulin administration, the calculation model may be structured to output an indication that a supply amount of insulin to the patient from outside of patient's body and a necessary amount are balanced, an indication that an insulin amount in the body is not sufficient, or an indication that the insulin amount in the body is excess, on the basis of the determination. As described above, the insulin is administered if the insulin in blood of the patient is not sufficient. Thus, determination of the dosage direction of STAY, UP or DOWN by the calculation model correspond to the state that the supply amount of insulin to the patient from outside of patient's body and the necessary amount are balanced, the state that the insulin amount in the body is not sufficient, and the state that the insulin amount in the body is excess, respectively. That is, the calculation model may be structured to output the indication that the supply amount of insulin to the patient from outside of patient's body and the necessary amount are balanced, the indication that the insulin amount in the body is not sufficient, or the indication that the insulin amount in the body is excess. For example, a doctor can find out a clinical state of the patient by looking at results of the output.

### Tenth Embodiment

Training data of a drug administration quantitative management assisting system 1 according to a tenth embodiment of the present invention further includes data of previous insulin administration amounts. The tenth embodiment is configured identically with the ninth embodiment except the above.

### Eleventh Embodiment

A calculator 11 of a drug administration quantitative management assisting system 1 according to an eleventh embodiment of the present invention is structured to calculate out, on the basis of the probabilities of the dosage direction of STAY, UP or DOWN obtained by the calculation model stored in a calculation model storage 12, a first determination for determining the dosage direction of STAY or NON-STAY, and a second determination for determining the dosage direction of UP or DOWN if the first determination is NON-STAY.

Operations of the calculator 11 of the drug administration quantitative management assisting system 1 according to the eleventh embodiment will be described with reference to FIG. 3.

At step S1, the calculator 11 obtains a probability P_{stay} of STAY, a probability Pᵤₚ of UP, and a probability P_{down} of DOWN in regard to the insulin administration determination from the calculation model stored in the calculation model storage 12. Note that P_{stay}+Pᵤₚ+P_{down}=1.

At step S2, the calculator 11 sets a threshold T for the first determination. Note that 0<T<1. T=0 means that the determination is always STAY, while T=1 always means that the determination is always UP or DOWN.

At step S3, the calculator 11 makes the first determination, that is, determines STAY or NON-STAY. More specifically, the calculator 11 determines whether or not P_{stay}≥T.

If the first determination is positive at step S3, the process goes to step S4.

At step S4, the calculator 11 outputs the result of the calculation that the insulin administration is determined to be STAY, and the process is ended.

If the first determination is negative at step S3, the process goes to step S5.

At step S5, the calculator 11 makes the second determination, that is, determines UP or DOWN. More specifically, the calculator 11 determines whether or not Pᵤₚ≥P_{down}.

If the second determination is positive at step S5, the process goes to step S6.

At step S6, the calculator 11 outputs the result of the calculation that the insulin administration is determined to be UP, and the process is ended.

If the second determination is negative at step S5, the process goes to step S7.

At step S7, the calculator 11 outputs the result of the calculation that the insulin administration is determined to be DOWN, and the process is ended.

According to the present embodiment, it is possible to realize a drug administration quantitative management assisting system capable of calculating out determination of insulin administration with a high accuracy.

### Twelfth Embodiment

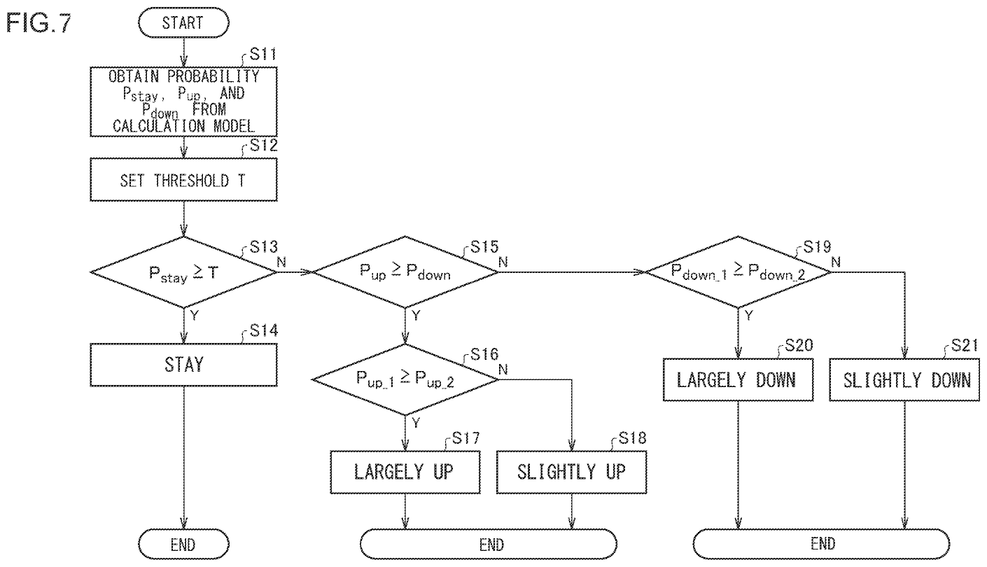

A drug administration quantitative management assisting system 1 according to a twelfth embodiment of the present invention will be described with reference to FIG. 7. The drug administration quantitative management assisting system 1 includes an inputter 10 and a calculator 11. The calculator 11 includes a calculation model storage 12.

The inputter 10 is structured to receive, as input data, blood sugar levels in blood, insulin administration amounts, and time periods passed from the end of the cardiovascular surgery to the insulin administration of a post-cardiovascular surgery patients. The input data is transmitted to the calculator 11.

The calculator 11 is structured to calculate out, from the input data received from the inputter 10, quinary determination of the insulin administration to the post-cardiovascular surgery patients as to the dosage direction of STAY, largely UP, slightly UP, largely DOWN or slightly DOWN with respect to the previous drug administration, on the basis of a calculation model stored in the calculation model storage 12. More specifically, the calculator 11 inputs, into the calculation model, the input data received from the inputter 10, thereby obtaining probabilities of the dosage direction of STAY, UP or DOWN for being determined as the determination of the insulin administration. The calculator 11 calculates out the quinary determination of the insulin administration as to the dosage direction of STAY, largely UP, slightly UP, largely DOWN or slightly DOWN on the basis of these probabilities.

It is known that the insulin administration management to the post-cardiovascular surgery patients would be more advantageous if the management was carried out more subtly than the drug administration management to chronic renal failure patients described in the first to eighth embodiments. In the present embodiment, the amount of the insulin administration to the post-cardiovascular surgery patients can be managed by 5 stages such as STAY, largely UP, slightly UP, largely DOWN or slightly DOWN. Thus, according to the present embodiment, it is possible to realize a drug administration quantitative management assisting system capable of calculating out determination of more subtle administration.

### Thirteenth Embodiment

A calculator 11 of a drug administration quantitative management assisting system 1 according to a thirteenth embodiment is structured to calculate out, on the basis of the probabilities of the dosage direction of STAY, UP or DOWN obtained from the calculation model stored in a calculation model storage 12, a first determination for determining the dosage direction of STAY or NON-STAY, a second determination for determining the dosage direction of UP or DOWN if the first determination is NON-STAY, a third determination for determining the dosage direction of largely UP, slightly UP if the second determination is UP, and a fourth determination for determining the dosage direction of largely DOWN, slightly DOWN if the second determination is DOWN.

FIG. 7 is a flow diagram illustrating an operation of a calculator 11 of the drug administration quantitative management assisting system 1 according to the thirteenth embodiment.

At step S11, the calculator 11 obtains a probability P_{stay} of STAY, a probability Pᵤₚ of UP, and a probability P_{down} of DOWN in regard to the insulin administration determination from the calculation model stored in the calculation model storage 12. Note that P_{stay}+Pᵤₚ+P_{down}=1.

At step S12, the calculator 11 sets a threshold T for the first determination. Note that 0<T<1. T=0 means that the determination is always STAY, while T=1 always means that the determination is always UP or DOWN.

At step S13, the calculator 11 makes the first determination, that is, determines STAY or NON-STAY. More specifically, the calculator 11 determines whether or not P_{stay}≥T.

If the first determination is positive at step S13, the process goes to step S14.

At step S14, the calculator 11 outputs the result of the calculation that the insulin administration is determined to be STAY, and the process is ended.

If the first determination is negative at step S13, the process goes to step S15.

At step S15, the calculator 11 makes the second determination, that is, determines UP or DOWN. More specifically, the calculator 11 determines whether or not Pᵤₚ≥P_{down}.

If the second determination is positive at step S15, the process goes to step S16.

At step S16, the calculator 11 makes the third determination, that is, determines largely UP or slightly UP, if the insulin administration is UP. More specifically, the calculator 11 determines whether or not P_{up_1}≥P_{up_2}. Here, P_{up_1} is the probabilities of the dosage direction of largely UP and P_{up_2} is the probabilities of the dosage direction of slightly UP, where P_{up_1}+P_{up_2}=Pᵤₚ.

If the third determination is positive at step S16, the process goes to step S17.

At step S17, the calculator 11 outputs a result of the determination of the dosage direction of largely UP, and the process ends.

If the third determination is negative at step S16, the process goes to step S18.

At step S18, the calculator 11 outputs a result of the determination of the dosage direction of slightly UP, and the process ends.

If the second determination is negative at step S15, the process goes to step S19.

At step S19, the calculator 11 makes the fourth determination, that is, determines largely DOWN or slightly DOWN, if the insulin administration is DOWN. More specifically, the calculator 11 determines whether or not P_{down_1}≥P_{down_2}. Here, P_{down_1} is the probabilities of the dosage direction of slightly DOWN, where P_{down_1}+P_{down_2}=P_{down}.

If the fourth determination is positive at step S19, the process goes to step S20.

At step S20, the calculator 11 outputs a result of the determination of the dosage direction of largely DOWN, and the process ends.

If the fourth determination is negative at step S19, the process goes to step S21.

At step S21, the calculator 11 outputs a result of the determination of the dosage direction of slightly DOWN, and the process ends.

According to the present embodiment, the amount of the insulin administration can be calculated by 5 stages such as STAY, largely UP, slightly UP, largely DOWN or slightly DOWN, thereby making it possible to realize a drug administration quantitative management assisting system capable of providing subtle management.

### Verification 4

In order to verify the applicability of the present invention to post-cardiovascular surgery patients, the determinations made by the drug administration quantitative management assisting system and determinations made by the medical specialists were compared with each other for 18 cases of actual patients by using changes of blood sugar level over time and data of amounts of insulin administration within 24 hours from surgeries. The results show that both the determinations match with each other about a 60% matching rate. This shows that the drug administration quantitative management assisting system is sufficiently applicable to blood sugar level administration to post-cardiovascular surgery patients.

So far, explanation has been made on the basis of some embodiments of the present invention. Person skilled in the art would understand that these embodiments are merely illustrative, various modifications and changes can be made within the scope of claims of the present invention, and that these modifications and changes are also within the scope of the present invention. Therefore, the description in this Description and Drawings should be considered as not limiting the present invention but merely for illustrative only.

For example, the drug administration quantitative management assisting system 2 in FIG. 4 may include a calculation model generator structured to prepare the calculation model by machine learning. By providing such updated new calculation model to the calculation model updater 13 periodically or as needed, the calculation model stored in the calculation model storage 12 may be updated. According to this modification, the calculation model can be prepared or updated within the drug administration quantitative management assisting system without providing the calculation model by externally preparing or updating the calculation model.

The modifications can bring about effects and advantages similar to those of embodiments.

Arbitrary combinations of embodiments and modifications are also applicable as embodiments of the present invention. New embodiments obtained by such combinations have the effects of embodiments and modifications thus combined.

### INDUSTRIAL APPLICABILITY

The present invention is applicable to a drug administration quantitative management assisting system.

### REFERENCE SIGNS LIST

1 drug administration quantitative management assisting system, 2 drug administration quantitative management assisting system, 10 inputter, 11 calculator, 12 calculation model storage, 13 calculation model updater, S3 first determination, S5 second determination, S13 first determination, S15 second determination, S16 third determination, S19 fourth determination.

1. A drug administration quantitative management assisting system, comprising:
an inputter structured to receive, as input data, a time passed from previous drug administration to a patient and/or a level of biological materials in blood of the patient and/or a change of the level; and
a calculator structured to calculate out from the input data, probabilities of drug administration to the patient as trinary determination of the dosage direction of STAY, UP or DOWN on the basis of a calculation model, and to calculate out a first determination for determining the dosage direction of STAY or NON-STAY on the basis of the calculated probabilities of drug administration, and a second determination for determining the dosage direction of UP or DOWN if the first determination is NON-STAY,
wherein the calculation model is prepared by machine learning by using, as training data, the time passed from previous drug administration to a plurality of patients and/or the value of the biological material in blood of the plurality of patient and/or the change of the value, and data indicating, as determination of the previous drug administration to the plurality of patients determined by doctors, any one of dosage directions of STAY, UP or DOWN.

2. The drug administration quantitative management assisting system according to claim 1, further comprising
a calculation model updater structured to update the calculation model to a new calculation model.

3. The drug administration quantitative management assisting system according to claim 1, wherein the training data further includes data indicating, as previous determination of the drug administration made by a doctor for the patient, any one of dosage directions of STAY, UP or DOWN.

4. The drug administration quantitative management assisting system according to claim 1, wherein the training data further includes data of amounts of previous drug administration.

5. The drug administration quantitative management assisting system according to claim 1, wherein the training data does not include data of patients who had been infected with an infectious disease or had a surgery.

6. The drug administration quantitative management assisting system according to claim 1,
wherein the patient is a chronic renal failure patient,
the value of the biological material in the blood includes a Hb level, a Ferritin level, and a TSAT level, and the change of the value is a change of the Hb level, and
a drug administered by the drug administration is at least one of an ESA formulation or an iron-containing agent.

7. The drug administration quantitative management assisting system according to claim 6, wherein the value of the biological material in the blood further includes an MCV level and the change of the value further includes a change of the MCV level.

8. The drug administration quantitative management assisting system according to claim 6, wherein the calculation model outputs an indication that a supply amount of EPO to the patient from outside of patient's body and a necessary amount are balanced, an indication that an EPO amount in the body is not sufficient, or an indication that the EPO amount in the body is excess, on the basis of the determination of the dosage direction of STAY, UP or DOWN.

9. The drug administration quantitative management assisting system according to claim 1, wherein the calculator calculates out a third determination for determining the dosage direction of largely UP or slightly UP if the second determination is UP, and a fourth determination for determining the dosage direction of largely DOWN or slightly DOWN if the second determination is DOWN.

10. The drug administration quantitative management assisting system according to claim 1, wherein the patient is a post-cardiovascular surgery patient,
the value of the biological materials in the blood is a blood sugar level, and
the drug administered by the drug administration is insulin.

11. The drug administration quantitative management assisting system according to claim 10, wherein the calculation model outputs an indication that a supply amount of insulin to the patient from outside of patient's body and a necessary amount are balanced, an indication that an insulin amount in the body is not sufficient, or an indication that the insulin amount in the body is excess, on the basis of the determination of the dosage direction of STAY, UP or DOWN.

12. A drug administration quantitative management assisting system, comprising:
an inputter structured to receive, as input data, input of an Hb level, an MCV level, a Ferritin level, a TSAT level, a change of the Hb level, and a change of the MCV level in blood of a chronic renal failure patient; and
a calculator structured to calculate out from the input data, probabilities of drug administration to the chronic renal failure patient as trinary determination of the dosage direction of STAY, UP or DOWN on the basis of a calculation model,
wherein the calculation model is prepared by machine learning by using, as training data, an Hb level, an MCV level, a Ferritin level, a TSAT level, a change of the Hb level, and a change of the MCV level in blood of a chronic renal failure patient, and data indicating, as determination of the previous drug administration to the plurality of patients determined by doctors, any one of dosage directions of STAY, UP or DOWN,
wherein the calculation model outputs probabilities of the dosage direction of STAY, UP or DOWN for being determined as the determination of the drug administration, and
a drug administered by the drug administration is at least one of an ESA formulation or an iron-containing agent.

13. The drug administration quantitative management assisting system according to claim 12, wherein the training data further includes data indicating, as previous determination of the drug administration made by a doctor for the patient, any one of dosage directions of STAY, UP or DOWN.

14. The drug administration quantitative management assisting system according to claim 12, wherein the training data further includes data of amounts of previous drug administration.

15. The drug administration quantitative management assisting system according to claim 12, wherein the training data includes data indicating whether the amount of the previous drug administration was 0 or not.

16. The drug administration quantitative management assisting system according to claim 12, wherein the calculator calculates out, on the basis of the probabilities of the dosage direction of STAY, UP or DOWN obtained by the calculation model, a first determination for determining the dosage direction of STAY or NON-STAY, and a second determination for determining the dosage direction of UP or DOWN if the first determination is NON-STAY.
